(19) United States
(12) Patent Application Publication
(10) Pub. No.: US 2026/0157848 A1
(43) Pub. Date: Jun. 11, 2026

(54) HAPTIC MANAGEMENT FOR SURGICAL IMPLANTS

(71) Applicant: Alcon Inc., Fribourg (CH)

(72) Inventors: Yinghui Wu, Cedar Hill, TX (US); Harlen Hoang, Fort Worth, TX (US); R. Mitchell Sherry, Fort Worth, TX (US); Jack Robert Auld, Laguna Niguel, CA (US); Matthew Braden Flowers, Laguna Niguel, CA (US); Matthew Douglas McCawley, Grant, FL (US); Andrew Thomas Schieber, St. Louis Park, MN (US); Marcus Antonio Souza, Costa Mesa, CA (US); Sudarshan B. Singh, Euless, TX (US); John Dunne, Irvine, CA (US); Quinton A. Quintana, Corona, CA (US)

(21) Appl. No.: 19/412,389

(22) Filed: Dec. 8, 2025

Related U.S. Application Data
(60) Provisional application No. 63/729,578, filed on Dec. 9, 2024.

(51) Int. Cl. A61F 2/16

(52) U.S. Cl. CPC A61F 2/1678; A61F 2/1691; A61F 2/167; A61F 2002/1681; A61F 2220/0008

(57) ABSTRACT

An apparatus for eye surgery may include an implant support, a folding arm, an edge roller, and a dial. Some embodiments of the implant support may include an implant bay, a tab rail, a tab slot, and a pawl. The folding arm may be coupled to the implant support and may include a dial pin. The edge roller may be coupled to the implant support and may include a roller track. Some embodiments of the dial may include an arm track, a roller pin, a plurality of ratchet teeth, and a tab. The tab can be configured to be aligned with the tab slot so that arm track engages the dial pin, and the roller pin engages the roller track. The dial can be configured to be rotated to move the tab behind the tab rail and to engage the ratchet teeth with the pawl.

### PRIORITY CLAIM

This application claims the benefit of priority of U.S. Provisional Patent Application Ser. No. 63/729,578 titled “HAPTIC MANAGEMENT FOR SURGICAL IMPLANTS,” filed on Dec. 9, 2024, whose inventors are Yinghui Wu, Harlen Hoang, R. Mitchell Sherry, Jack Robert Auld, Matthew Braden Flowers, Matthew Douglas McCawley, Andrew Thomas Scheiber, Marcus Antonio Souza, Sudarshan B. Singh, John Dunne and Quinton A. Quintana, which is hereby incorporated by reference in its entirety as though fully and completely set forth herein.

### TECHNICAL FIELD

The invention set forth in the appended claims relates generally to eye surgery. More particularly, but without limitation, the claimed subject matter relates to systems, apparatuses, and methods for inserting an implant into an eye.

### BACKGROUND

The human eye can suffer a number of maladies causing mild deterioration to complete loss of vision. While contact lenses and eyeglasses can compensate for some ailments, ophthalmic surgery may be required for others. In some instances, implants may be beneficial or desirable. For example, an intraocular lens may replace a clouded natural lens within an eye to improve vision.

While the benefits of intraocular lenses and other implants are known, improvements to delivery systems, components, and processes continue to improve outcomes and benefit patients.

### BRIEF SUMMARY

New and useful systems, apparatuses, and methods for eye surgery are set forth in the appended claims. Illustrative embodiments are also provided to enable a person skilled in the art to make and use the claimed subject matter.

For example, some embodiments may relate to an apparatus for eye surgery that can be readily assembled in a secure and stable configuration for shipping and storage. More particular examples may include a mechanical control, such as a dial, that can be coupled to an implant support with a single turn and can also provide tactile and/or audible feedback to indicate proper assembly.

In some examples, the dial may have tabs that can be aligned with tab openings in the implant support to reliably position the dial for installation. The dial may also have tracks configured to engage pins on one or more haptic folding arms in such a position. Additionally, or alternatively, some embodiments of the apparatus may have a ratchet mechanism to facilitate assembly. For example, the implant support may have a pawl, and the dial may have ratchet teeth configured to engage the pawl. Additionally, or alternatively, the implant support may have one or more springs that can engage the dial for stability. For example, each of the springs may be aligned with or engage a pocket or recess in the dial. The dial can be turned so that the tabs engage a tab rail on the implant support. As the dial turns, the springs disengage from one pocket and drop into another pocket while the pawl simultaneously engages the first tooth of the ratchet teeth. The action of the springs and/or the pawl can provide tactile and audible feedback. Additionally, or alternatively, the springs and/or the pawl can prevent the dial from being turned in the opposite direction once installed, thereby locking the dial in a stable position for shipping and storage.

More generally, some embodiments may relate to an apparatus for managing an implant to be delivered to an eye, and the apparatus may include an implant support, a folding arm, an edge roller, and a dial. Some embodiments of the implant support may include an implant bay, a tab rail, a tab slot, and a pawl. The folding arm may be configured to be coupled to the implant support and may include a dial pin. The edge roller may be configured to be coupled to the implant support and may include a roller track. Some embodiments of the dial may include an arm track, a roller pin, a plurality of ratchet teeth, and a tab. The tab can be configured to be aligned with the tab slot in the implant support so that the arm track engages the dial pin, and the roller pin engages the roller track. The dial can be configured to be rotated to move the tab behind the tab rail and to engage the ratchet teeth with the pawl. In some embodiments, the tab rail may be disposed around the implant bay, and the tab slot and the pawl are substantially co-planar with the tab rail.

Some embodiments may relate to an apparatus for eye surgery, which may include an implant support, a first folding arm, a second folding arm, a first edge roller, a second edge roller, and a dial. Some embodiments of the implant support may include an implant bay, a tab rail, a plurality of tab slots, and a pawl. In some embodiments, an implant may be disposed in the implant bay, and the implant may include an optic body, a first haptic coupled to the optic body, and a second haptic coupled to the optic body. The first folding arm may be configured to be coupled to the implant support and may include a first dial pin and a first haptic tab. The first haptic tab can be configured to engage the first haptic. The second folding arm may be configured to be coupled to the implant support and may include a second dial pin and a second haptic tab. The second haptic tab can be configured to engage the second haptic. The first edge roller may be configured to be coupled to the implant support and may include a first roller track and a first folding edge. The first folding edge can be configured to engage a first portion of the optic body. The second edge roller can be configured to be coupled to the implant support and may include a second roller track and a second folding edge. The second folding edge can be configured to engage a second portion of the optic body. The dial may include a ring, a first arm track, a second arm track, a first roller pin, a second roller pin, a plurality of ratchet teeth, and a plurality of tabs. The ring can circumscribe the tab rail, and each of the tabs may be configured to be aligned with one of the tab slots so that the first arm track engages the first dial pin, the second arm track engages the second dial pin, the first roller pin engages the first roller track, the second roller pin engages the second roller track. The dial can be configured to be rotated to move the tabs behind the tab rail and to engage a first tooth of the ratchet teeth with the pawl.

In other aspects, some embodiments may relate to a method for assembling an apparatus for eye surgery. The method may generally comprise or consist essentially of: receiving an implant support, which may include an implant bay, a tab rail, a tab slot, and a pawl; receiving a folding arm, which may include a dial pin; receiving an edge roller, which may include a roller track; receiving a dial, which may include an arm track, a roller pin, a plurality of ratchet teeth, and a tab; coupling the folding arm and the edge roller to the implant support; aligning the tab with the tab slot so that the arm track engages the dial pin and the roller pin engages the roller track; and rotating the dial to simultaneously move the tab behind the tab rail and engage the ratchet teeth with the pawl.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is an isometric view of an example apparatus for delivering an implant into an eye.

FIG. 2 is an auxiliary view of the apparatus of FIG. 1.

FIG. 3 is a section view of the apparatus of FIG. 1.

FIG. 4 is a front view of a housing that may be associated with the apparatus of FIG. 1.

FIG. 5 is a side view of the housing of FIG. 4.

FIG. 6 is a top view of an example of a plunger that may be associated with the apparatus of FIG. 1.

FIG. 7 is an assembly view of an implant management system that may be associated with the apparatus of FIG. 1.

FIG. 8 is a top view of an implant support that may be associated with the implant management system of FIG. 7.

FIG. 9 is a section view of the implant support of FIG. 8.

FIG. 10 is an isometric view of an example of a folding arm that may be associated with the implant management system of FIG. 7.

FIG. 11 is an isometric view of an example of an edge roller that may be associated with the implant management system of FIG. 7.

FIG. 12 is a bottom view of a dial that may be associated with the implant management system of FIG. 7.

FIG. 13 is a top view of the implant management system of FIG. 7 in an initial state of assembly.

FIG. 14 is a top view of the implant management system of FIG. 13, wherein a portion of the dial has been removed to illustrate additional details.

FIG. 15 is another top view of the implant management system of FIG. 13, wherein the cap has been removed to illustrate additional details.

FIG. 16 illustrates the implant management system of FIG. 14 in a second state, in which the dial is in an assembled and locked position.

FIG. 17 illustrates the implant management system of FIG. 15 in the second state.

FIG. 18 illustrates the implant management system of FIG. 17 in a third state, in which the dial has been rotated further.

FIG. 19 illustrates the implant management system of FIG. 18, with an additional portion of the dial removed to show additional features.

FIG. 20 illustrates the implant management system of FIG. 19 in a fourth state, in which the dial has been rotated further.

FIG. 21 illustrates the implant management system in the fourth state of FIG. 20, in which an additional portion of the dial has been removed to illustrate further details.

FIG. 22 is a section view of the implant management system in the fourth state.

FIG. 23A and FIG. 23B are section views of the apparatus of FIG. 3.

FIG. 24 is a schematic diagram illustrating an example method of using the apparatus of FIG. 1 to eject an implant.

FIG. 25 and FIG. 26 are each an isometric view of another example of an apparatus for delivering an implant into an eye.

FIG. 27A and FIG. 27B are schematic diagrams illustrating an example use of the apparatus to deliver an implant to an eye.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive an implant. However, as should be recognized by those skilled in the art, this frame of reference is merely a descriptive expedient rather than a strict prescription.

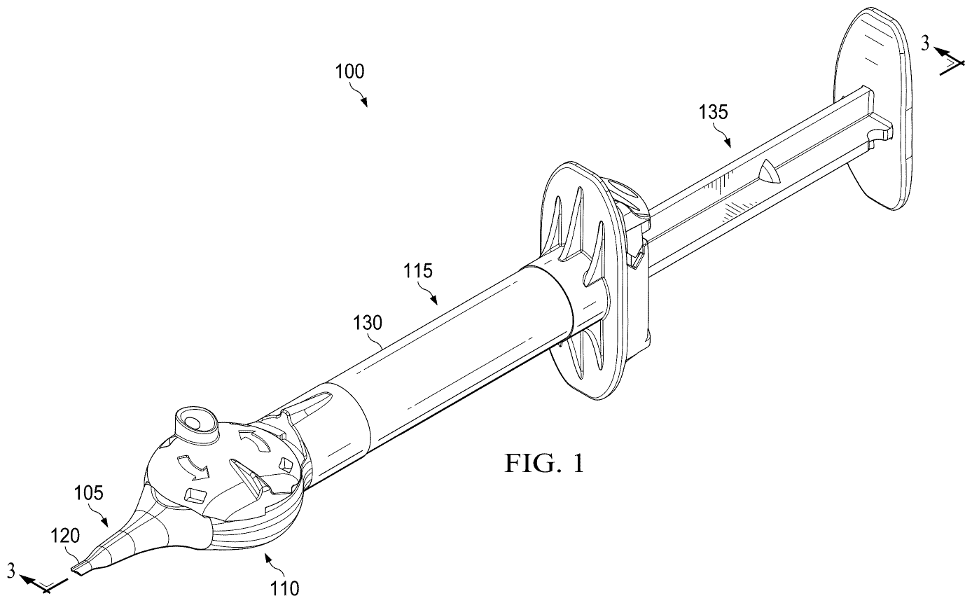

FIG. 1 is an isometric view of an example of an apparatus 100 that can deliver an implant into an eye. In some embodiments, the apparatus 100 may comprise two or more modules, which can be configured to be coupled and decoupled as appropriate for storage, assembly, use, and disposal. As illustrated in FIG. 1, some embodiments of the apparatus 100 may include a nozzle 105, an implant management system 110 coupled to the nozzle 105, and an actuator 115 coupled to the implant management system 110.

The nozzle 105 generally comprises a tip 120 adapted for insertion through an incision into an eye. The size of the tip 120 may be adapted to surgical requirements and techniques as needed. For example, small incisions are generally preferable to reduce or minimize healing times. Incisions of less than 2 millimeters may be preferable in some instances, and the tip 120 of the nozzle 105 may have a width of less than 2 millimeters in some embodiments.

The implant management system 110 generally represents a wide variety of apparatuses that are suitable for storing an implant prior to delivery into an eye. In some embodiments, the implant management system 110 may additionally or alternatively be configured to prepare an implant for delivery. For example, some embodiments of the implant management system 110 may be configured to be actuated by a surgeon or other operator to prepare an implant for delivery by subsequent action of the actuator 115. In some instances, the implant management system 110 may be configured to actively deform, elongate, extend, or otherwise manipulate features of the implant before the implant is advanced into the nozzle 105. For example, the implant management system 110 may be configured to fold, tuck, extend or splay one or more features, such as haptics, of an intraocular lens.

The actuator 115 is generally configured to advance an implant from the implant management system 110 into the nozzle 105, and thereafter from the nozzle 105 through an incision and into an eye. The actuator 115 of FIG. 1 generally comprises a housing 130 and a plunger 135. The plunger 135 is generally comprised of a substantially rigid material, such as a medical grade polymer material.

In general, components of the apparatus 100 may be coupled directly or indirectly. For example, the nozzle 105 may be directly coupled to the implant management system 110 and may be indirectly coupled to the actuator 115 through the implant management system 110. Coupling may include fluid, mechanical, thermal, electrical, or chemical coupling (such as a chemical bond), or some combination of coupling in some contexts. For example, the implant management system 110 may be mechanically coupled to the actuator 115 and may be mechanically and fluidly coupled to the nozzle 105. In some embodiments, components may also be coupled by virtue of physical proximity, being integral to a single structure, or being formed from the same piece of material.

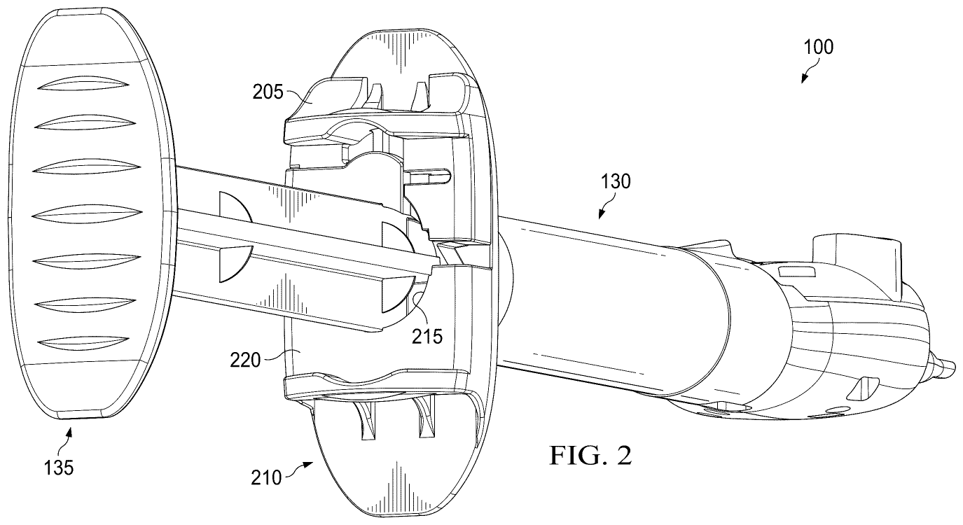

FIG. 2 is an auxiliary view of the apparatus 100, illustrating additional details that may be associated with some embodiments. For example, the apparatus 100 of FIG. 2 further comprises a plunger key 205, which may be coupled to a key mount 210 on the housing 130. The housing 130 generally has a bore 215 configured to receive the plunger 135 through the key mount 210. Some embodiments of the key mount 210 may further comprise a key box 220 configured to receive a portion of the plunger key 205, as illustrated in the example of FIG. 2.

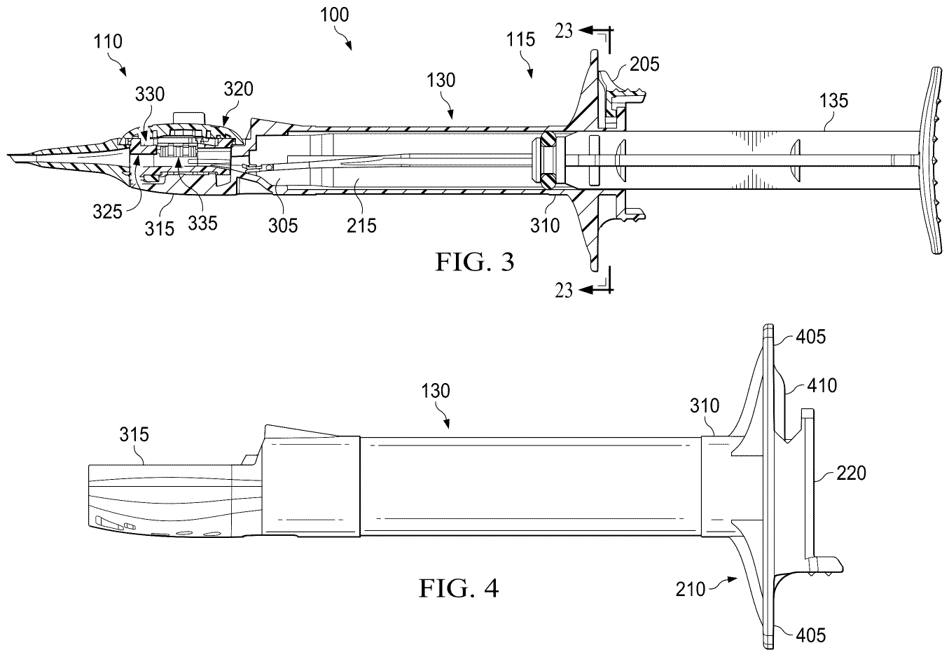

FIG. 3 is a section view of the apparatus 100 of FIG. 1, illustrating additional details that may be associated with some embodiments. For example, the bore 215 of FIG. 3 generally passes through the housing 130 longitudinally from a first end 305 to a second end 310, and the plunger 135 is disposed at least partially within the bore 215. In the example configuration of FIG. 3, the plunger key 205 is in a locked position, preventing the plunger 135 from moving through the bore 215.

In some examples, the implant management system 110 may comprise a base 315, a dial 320, an implant support 325, one or more folding arms 330, and one or more edge rollers 335. As illustrated in the example of FIG. 3, the dial 320 may be coupled to the base 315, and the implant support 325 can be disposed between the base 315 and the dial 320. In some examples, the implant support 325 may be coupled to the base 315, and the folding arms 330 and the edge rollers 335 may be coupled to the implant support 325. The base 315 may extend from or be coupled to actuator 115 in some examples. In FIG. 3, for example, the base 315 is coupled to the housing 130 proximate to the first end 305.

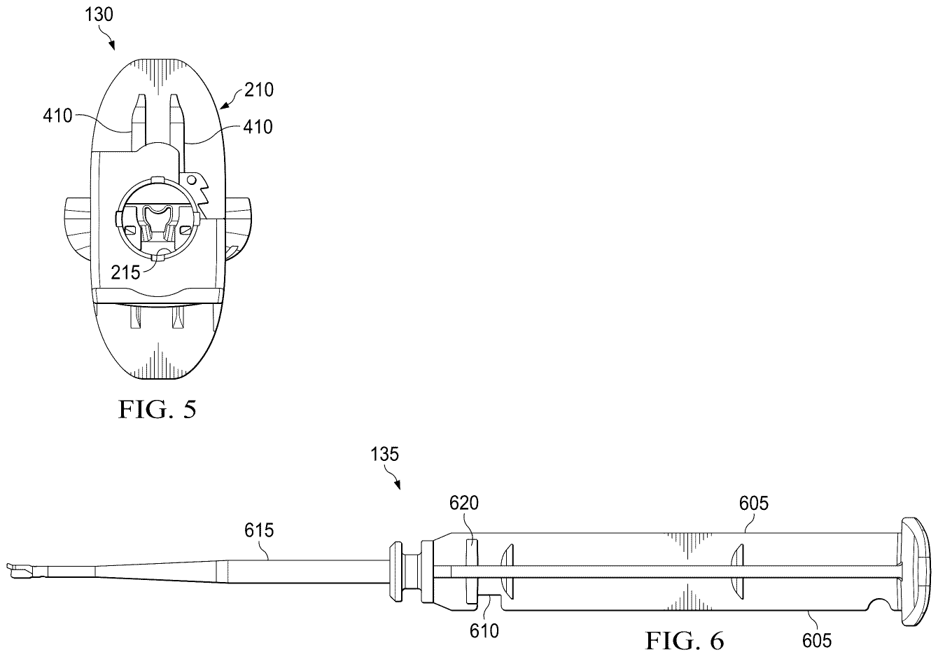

FIG. 4 is a front view of the housing 130 of FIG. 1, illustrating additional details that may be associated with some embodiments. In some embodiments, the key mount 210 may be coupled to the second end 310 of the housing 130. For example, the key mount 210 of FIG. 4 generally comprises a flange 405, which is coupled to the second end 310 of the housing 130. The key mount 210 may additionally comprise a key track 410. In some embodiments, the key track 410 may comprise or consist essentially of a ridge or rail, as in the example of FIG. 4. In other examples, the key track 410 may comprise or consist essentially of a channel or groove in the key mount 210. As shown in the example of FIG. 4, some embodiments of the key box 220 may be coupled to and extend from the flange 405. In the example of FIG. 4, the base 315 is shown as an integral extension of the housing 130; in other examples, the base 315 may be mechanically coupled to the housing 130.

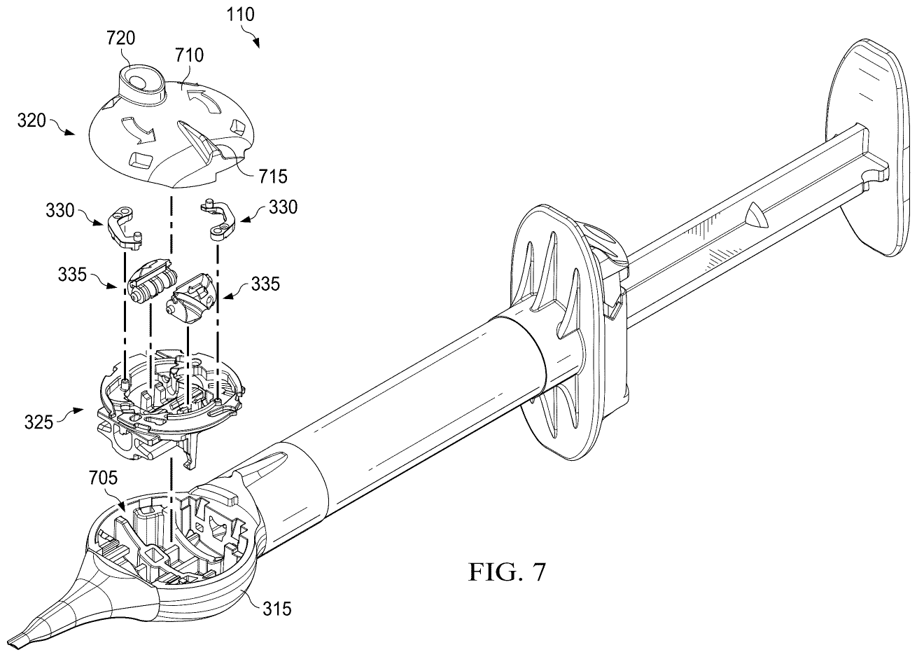

FIG. 5 is a side view of the housing 130 of FIG. 4, illustrating additional details that may be associated with some embodiments. For example, some embodiments of the key mount 210 may have more than one key track 410. In the example of FIG. 4, the key mount 210 comprises a first key track 410 and a second key track 410, which are mutually parallel to each other and generally orthogonal to the longitudinal direction of the bore 215.

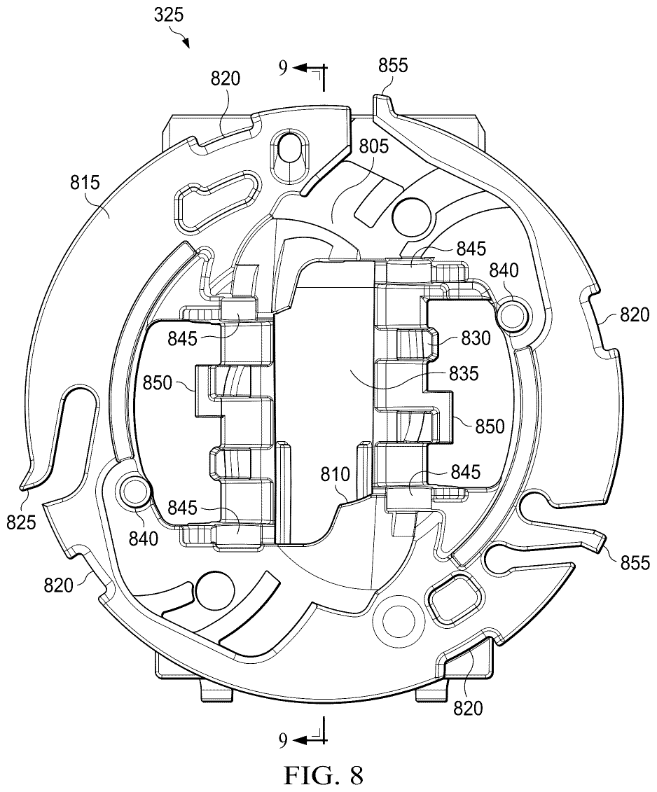

FIG. 6 is a top view of an example of the plunger 135, illustrating additional details that may be associated with some embodiments. As illustrated in the example of FIG. 6, the plunger 135 may comprise one or more ridges, rails, or similar features, such as a rail 605, and a keyway 610. In general, each of the rails runs lengthwise along a portion of the plunger 135. In the example of FIG. 6, one end of the plunger 135 further comprises an implant interface 615, and the rail 605 runs lengthwise along the plunger 135 between the implant interface 615 and the opposite end of the plunger 135. In some examples, the keyway 610 may be disposed in one of the rails. For example, the keyway 610 may be a channel, groove, slot, notch, or similar feature in one of the rails. As show in FIG. 6, some embodiments of the keyway 610 may be disposed through the rail 605. Some embodiments of the plunger 135 may additionally comprise a plunger stop, such as a stop 620 illustrated in the example of FIG. 6.

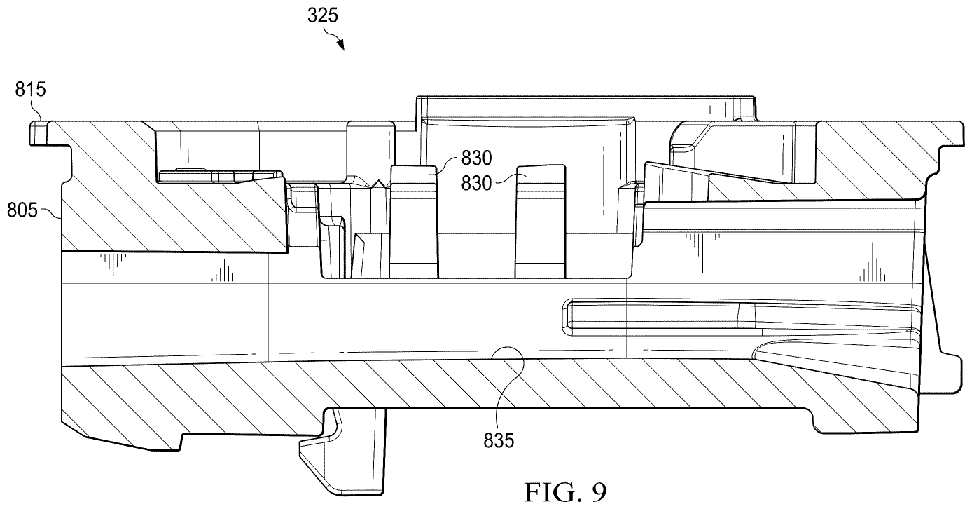

FIG. 7 is an assembly view of the implant management system 110 of FIG. 1, including the base 315, the dial 320, the implant support 325, the folding arms 330, and the edge rollers 335, illustrating additional details that may be associated with some embodiments. For example, the base 315 of FIG. 7 defines a cavity 705 into which the implant support 325 may be inserted. In some examples, the implant support 325 may be configured to be snapped into the base 315 to mechanically couple the implant support 325 to the base 315. The implant management system 110 of FIG. 7 comprises two of the folding arms 330 and two of the edge rollers 335. As illustrated in the example of FIG. 7, the dial 320 may include a cap 710, an actuator tab 715 coupled to the cap 710, and a fill port 720 coupled to the cap 710.

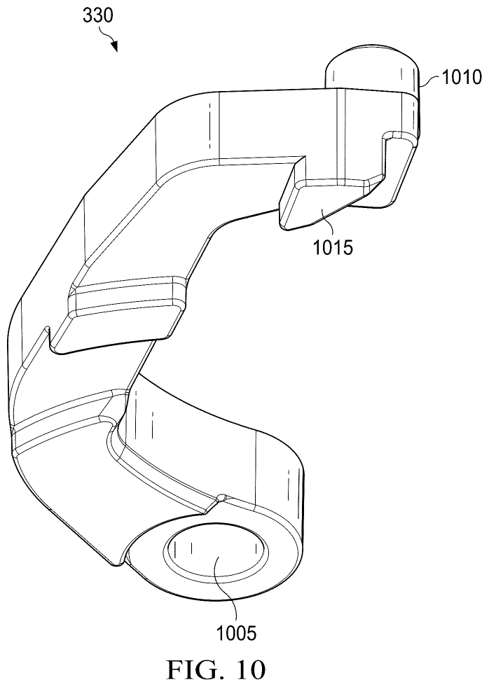

FIG. 8 is a top view of the implant support 325 of FIG. 7, illustrating additional details that may be associated with some embodiments. For example, the implant support 325 of FIG. 8 comprises a frame 805, an implant bay 810, a tab rail 815, one or more tab slots 820, and a pawl 825. The implant bay 810 may be defined by the frame 805, and the tab rail 815 may be coupled to the frame 805. One or more optic supports 830 and a delivery channel 835 may be disposed below the implant bay 810 of FIG. 8. Additionally, some embodiments of the implant support 325 may comprise one or more pivot posts 840, pin slots 845, roller tabs 850, and spring arms 855. For example, the pivot posts 840, roller tabs 850, and spring arms 855 may be coupled to the frame 805, and the pin slots 845 may be recessed into the frame 805, as shown in FIG. 8. In some embodiments, each of the spring arms 855 may be a cantilever spring or other flexible arm having one end coupled to the frame 805 and one flexible free end.

At least a portion of the frame 805 may have a circular portion, as illustrated in the example of FIG. 8. Additionally, or alternatively, some embodiments of the tab rail 815 may be circular. In some examples, the tab rail 815 may be disposed at least partially around the implant bay 810, and the tab slots 820, the pawl 825, and the spring arms 855 may be disposed in the same plane defined by the tab rail 815. In other examples, the tab slots 820 may define a plane, which may be co-planar with a plane defined by the tab rail 815. In more particular examples, the tab slots 820 may be formed in the tab rail 815, and the pawl 825 and the spring arms 855 may be coupled to the tab rail 815, as illustrated in FIG. 8.

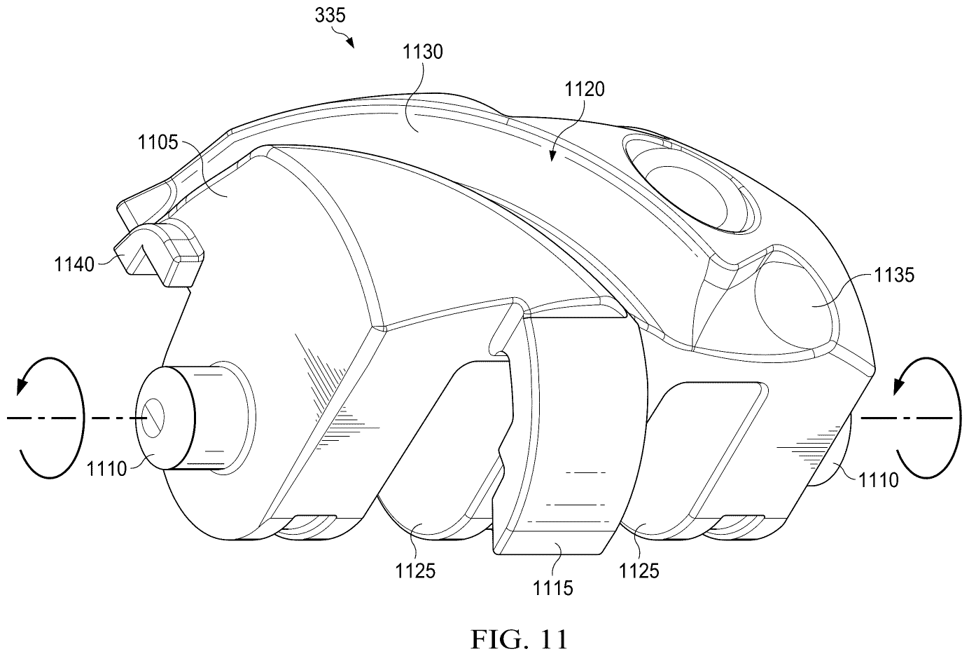

FIG. 9 is a section view of the implant support 325 taken along line 9-9 of FIG. 8, illustrating additional details that may be associated with some embodiments. For example, as illustrated in FIG. 9, the optic supports 830 and the delivery channel 835 may be coupled to or defined by the frame 805. FIG. 9 also illustrates the tab rail 815 coupled to a top portion of the frame 805.

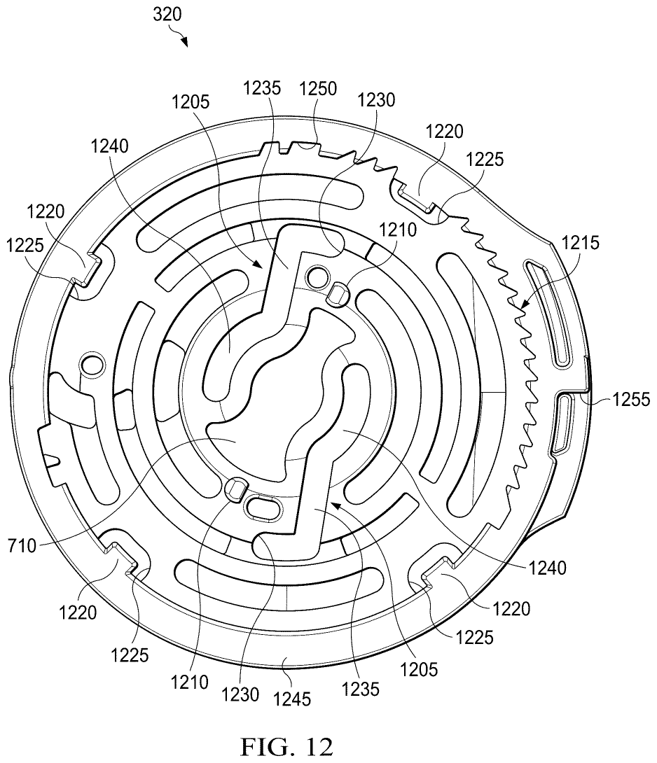

FIG. 10 is an isometric view of an example of the folding arms 330, illustrating additional details that may be associated with some embodiments. For example, some embodiments of the folding arms 330 may include bore 1005, a dial pin 1010, and a haptic tab 1015. As shown in FIG. 10, the bore 1005 may be disposed in a first end, and the dial pin 1010 and the haptic tab 1015 may be disposed at a second end. In some examples, the bore 1005 can define an axis of rotation for the folding arms 330. The dial pin 1010 and the haptic tab 1015 may extend from the second end in generally opposite directions.

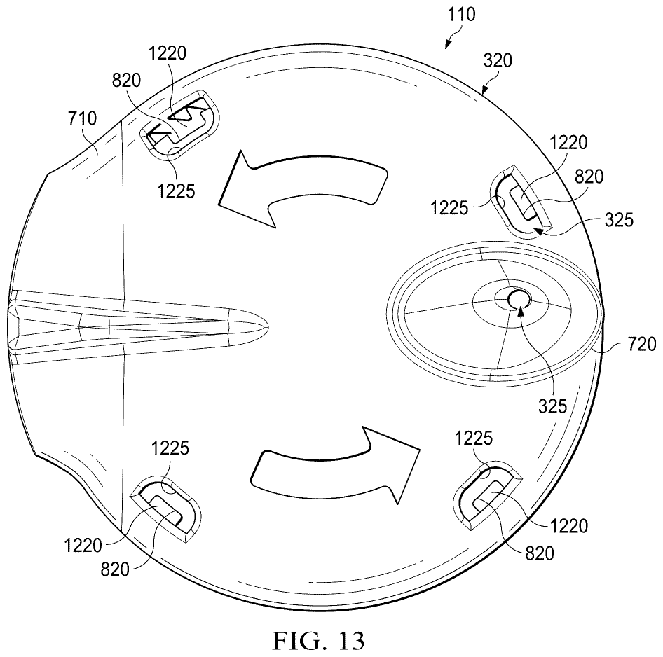

FIG. 11 is an isometric view of an example of the edge rollers 335, illustrating additional details that may be associated with some embodiments. For example, the edge roller 335 of FIG. 11 comprises a body 1105, pins 1110 coupled to the body 1105, a clip 1115 coupled to the body 1105, and roller track 1120 disposed in the body 1105. Some examples may also include one or more slots 1125 in the body 1105. The pins 1110 are generally disposed at opposing ends of the body 1105 and define an axis of rotation for the edge roller 335. In some examples, the roller track 1120 may be a groove or channel in the body 1105, as illustrated in FIG. 11. Additionally, the roller track 1120 may comprise more than one segment. For example, the roller track 1120 of FIG. 11 comprises a folding segment 1130 and a follower segment 1135, each having a different orientation relative to the axis of rotation. For example, the folding segment 1130 may be oriented at an acute angle relative to the axis of rotation, and the follower segment 1135 may be substantially parallel to the axis of rotation. In some examples, the folding segment 1130 may be oriented at an angle of approximately forty-five degrees relative to the axis of rotation. As shown in FIG. 11, the edge roller 335 may also have one or more folding features, such as a folding edge 1140.

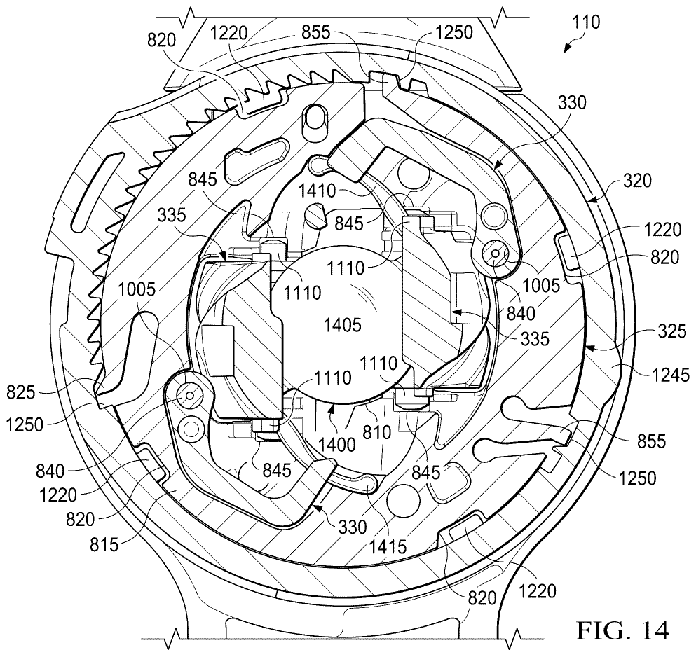

FIG. 12 is a bottom view of the dial 320 of FIG. 7, illustrating additional details that may be associated with some embodiments. For example, the dial 320 of FIG. 7 comprises a combination of tracks, pins, ratchets, and other features, including one or more arm tracks 1205, one or more roller pins 1210, a plurality of ratchet teeth 1215, and one or more tabs 1220. Some examples of the dial 320 may also comprise one or more tab windows 1225, where the number of tab windows 1225 is the same as the number of tabs 1220, and each of the tab windows 1225 is aligned with one of the tabs 1220. In some embodiments, the features may be molded into or coupled to the cap 710. For example, tracks may comprise channels, grooves, or other pathways molded into the cap 710. In some examples, some features may have symmetric properties. For example, the dial 320 of FIG. 12 comprises a pair of the arm tracks 1205 that are substantially symmetric and a pair of the roller pins 1210 that are substantially symmetric.

Each of the arm tracks 1205 may comprise more than one segment. For example, each of the arm tracks 1205 of FIG. 12 comprise a holding segment 1230, a folding segment 1235, and a follower segment 1240. The dial 320 of FIG. 12 also comprises a ring 1245, which generally circumscribes or encircles other features. In some examples, the ratchet teeth 1215 and the tabs 1220 may be coupled to the ring 1245. The ring 1245 of FIG. 12 includes one or more recesses 1250. Some embodiments of the dial 320 may also include a stop tab 1255. The ratchet teeth 1215 and the tabs 1220 of FIG. 12 are generally co-planar with a plane defined by the ring 1245.

Some embodiments of the holding segment 1230 and the follower segment 1240 may each be generally defined, at least in part, as a curved track that is substantially concentric with the ring 1245, and the folding segment 1235 may be a linear track that is transverse to the holding segment 1230, the follower segment 1240, or both.

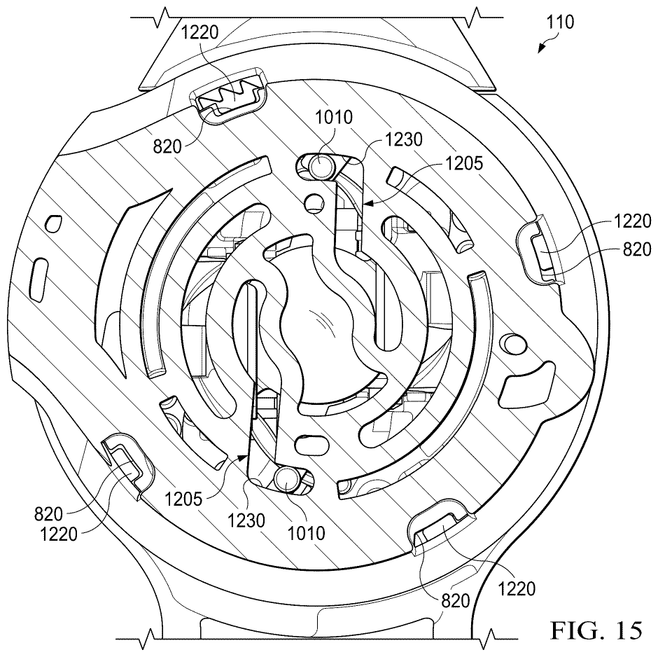

FIG. 13 is a top view of the implant management system 110 of FIG. 7 in an initial state of assembly, illustrating additional details that may be associated with some embodiments. In the initial state of assembly of FIG. 13, the dial 320 is disposed on the implant support 325 so that the tabs 1220 are aligned with the tab slots 820 in the implant support 325. As illustrated in FIG. 13, the tab windows 1225 may extend through the cap 710, and the tabs 1220 may be visible through the tab windows 1225 in this state of assembly, allowing the tabs 1220 to be visually aligned with the tab slots 820. In this state, the fill port 720 may be blocked by a portion of the implant support 325.

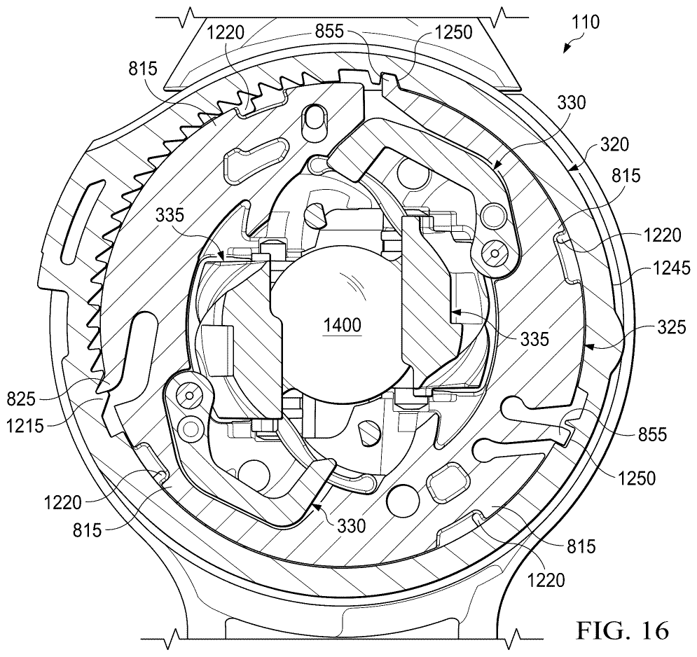

FIG. 14 is a top view of the implant management system 110 of FIG. 13, wherein a portion of the dial 320 has been removed to illustrate additional details that may be associated with some embodiments. For example, the ring 1245 of FIG. 14 circumscribes a top portion of the implant support 325. The ring 1245 of FIG. 14 is generally co-planar with the tab rail 815. In some examples, the ring 1245 may circumscribe the tab rail 815, so that portions of the ring 1245 may interface with an edge of the tab rail 815.

Additionally, the folding arms 330 and the edge rollers 335 are coupled to the implant support 325 in FIG. 14. In the example of FIG. 14, the bore 1005 of each of the folding arms 330 is rotatably mounted onto one of the pivot posts 840 of the implant support 325, and the pins 1110 of each of the edge rollers 335 is disposed in one of the pin slots 845. In this example configuration, the axes of rotation for the folding arms 330 and the axes of rotation for the edge rollers 335 are skew lines. Additionally, or alternatively, the folding arms 330 may rotate in a plane that is parallel to the plane defined by the ring 1245, and the edge rollers 335 may rotate out of plane with the ring 1245. In some examples, the edge rollers 335 may rotate substantially orthogonal to the rotation of the folding arms 330. Not visible in FIG. 14, each of the optic supports 830 may be inserted into one of the corresponding slots 1125 of the edge rollers 335, and the clip 1115 may engage the roller tabs 850 to provide initial resistance to rotation of the edge rollers 335.

FIG. 14 also includes an example of an implant 1400 disposed in the implant bay 810 in an initial state. In some embodiments, the implant 1400 may be an ophthalmic implant, such as an intraocular lens having a shape similar to that of a natural lens of an eye. The implant 1400 may be made from numerous materials. Examples of suitable materials may include silicone, acrylic, and combinations of such suitable materials. In some instances, the implant 1400 may comprise an intraocular lens that is fluid-filled, such as a fluid-filled accommodating intraocular lens. The implant 1400 may also comprise an intraocular lens that includes one or more features, such as haptics, for positioning the intraocular lens within an eye. For example, the implant 1400 of FIG. 14 is an intraocular lens having an optic body 1405, a leading haptic 1410, and a trailing haptic 1415.

In the initial state illustrated in FIG. 14, the tabs 1220 are aligned with the tab slots 820 in the implant support 325, substantially as illustrated in FIG. 13, and the pawl 825 and spring arms 855 are each engaged with one of the recesses 1250 in the ring 1245. The haptic tab 1015 (not visible in FIG. 14) on each of the folding arms 330 is engaged with the leading haptic 1410 or the trailing haptic 1415, and the folding edge 1140 of each of the edge rollers 335 is engaged with a portion of the optic body 1405.

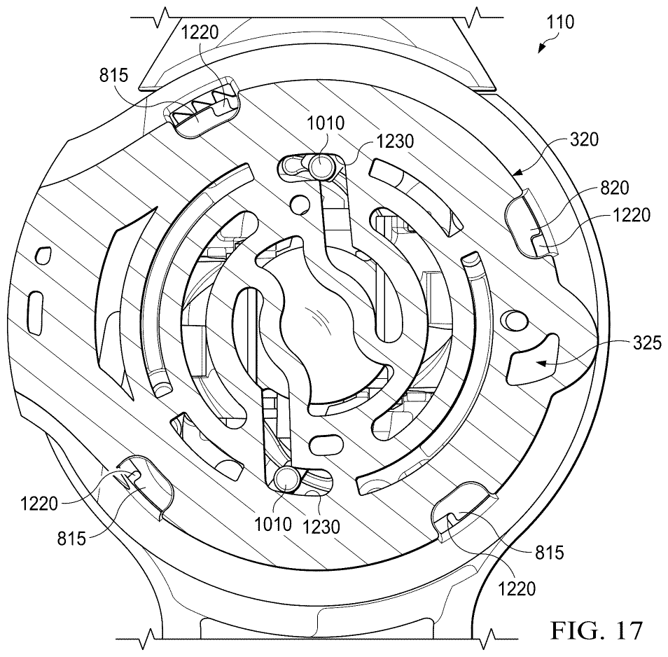

FIG. 15 is another top view of the implant management system 110 of FIG. 13, wherein a portion of the dial 320 has been removed to illustrate additional details that may be associated with some embodiments. For example, each of the dial pins 1010 may be engaged with one of the arm tracks 1205. In more specific examples, the dial pins 1010 may be engaged with one of the holding segments 1230 in the initial state, as illustrated in FIG. 15. As in FIG. 14, the tabs 1220 of FIG. 15 may be aligned with the tab slots 820.

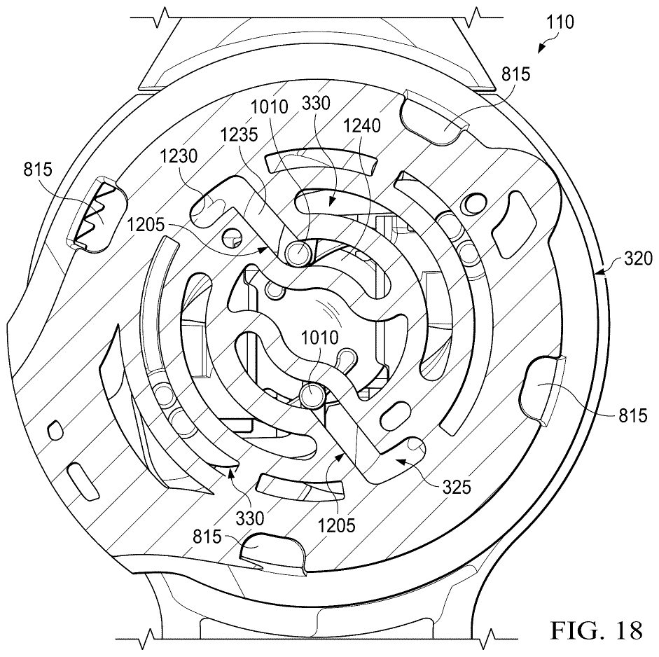

FIG. 16 illustrates the implant management system 110 of FIG. 14 in a second state, in which the dial 320 is in an assembled and locked position. In this state, the dial 320 has been rotated in a first direction (e.g., counterclockwise in this example) relative to the implant support 325. For example, the dial 320 may be rotated relative to the tab rail 815 so that the tabs 1220 rotate behind the tab rail 815, which can couple the dial 320 to the implant support 325. Additionally, rotation of the dial 320 can also simultaneously rotate the ratchet teeth 1215 relative to the pawl 825 and rotate the recesses 1250 relative to the spring arms 855. In the example state of FIG. 16, rotating the dial 320 has caused the pawl 825 to engage a first tooth in the ratchet teeth 1215, preventing rotation of the dial 320 in a second direction, opposite to the first direction. In some embodiments, rotation of the recesses 1250 can cause one or more of the spring arms 855 to flex out of one of the recesses 1250 and to drop into another of the recesses 1250. As illustrated in the example of FIG. 16, at least some of the recesses 1250 may be grouped into pairs of adjacent recesses 1250, where the number of pairs is generally the same as the number of spring arms 855. Rotating the dial 320 can cause the spring arms 855 to move from one of the adjacent recesses 1250 into the other. Each of these features, individually or collectively, allows the dial 320 to be installed onto the implant support 325 with a single “click”. For example, once the dial 320 is placed onto the implant support 325 in the initial state, substantially as illustrated in FIG. 13, rotating the dial 320 can cause the first tooth of the ratchet teeth 1215 to slide past the pawl 825, which can provide tactile and/or audible feedback. Movement of the spring arms 855 into the adjacent recesses 1250 can provide similar feedback. The folding arms 330, the edge rollers 335, and the implant 1400 of FIG. 16 are unchanged from the initial state of FIG. 14.

In this second state of FIG. 16, one or more of the features of the implant management system 110 can allow the dial 320 to be retained in a stable position prior to use. For example, the tabs 1220 of FIG. 16 are coupled to the tab rail 815, which can secure the dial 320 to the implant support 325. Additionally, or alternatively, the pawl 825 and the ratchet teeth 1215 can provide sufficient rotational resistance to substantially reduce or prevent unintentional rotation. The spring arms 855 and the recesses 1250 can also provide stability and rotational resistance. The number of the spring arms 855 can vary. In some examples, two of the spring arms 855 may be advantageous. It may also be advantageous to equidistantly space the pawl 825 and the spring arms 855 around the implant support 325. Similarly, it may be advantageous to space the recesses 1250 equidistantly around the ring 1245.

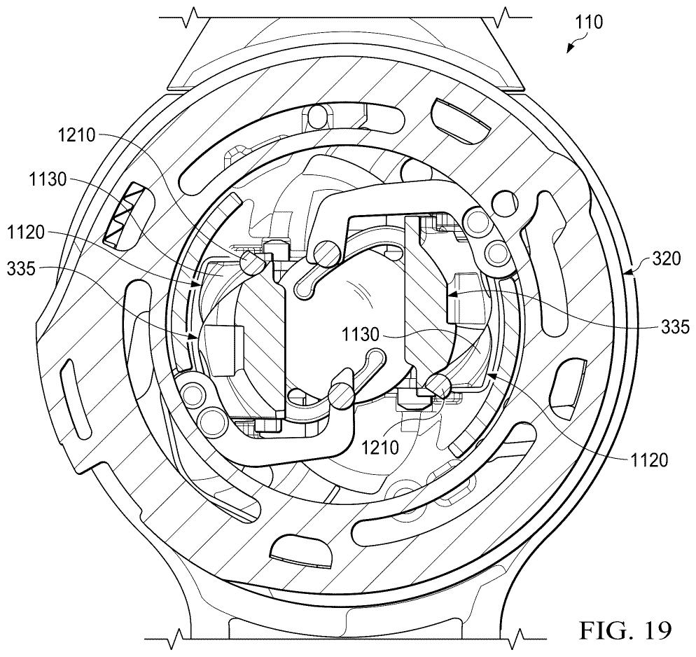

FIG. 17 illustrates the implant management system 110 of FIG. 15 in the second state, in which the dial 320 is in an assembled and locked position. As in FIG. 16, the dial 320 has been rotated in the first direction relative to the implant support 325, so that the tabs 1220 rotate behind the tab rail 815 to couple the dial 320 to the implant support 325. Additionally, the holding segments 1230 can allow the dial 320 to be rotated to the second state without changing the configuration of the dial pins 1010, which can remain engaged with the holding segments 1230 as illustrated in the example of FIG. 17.

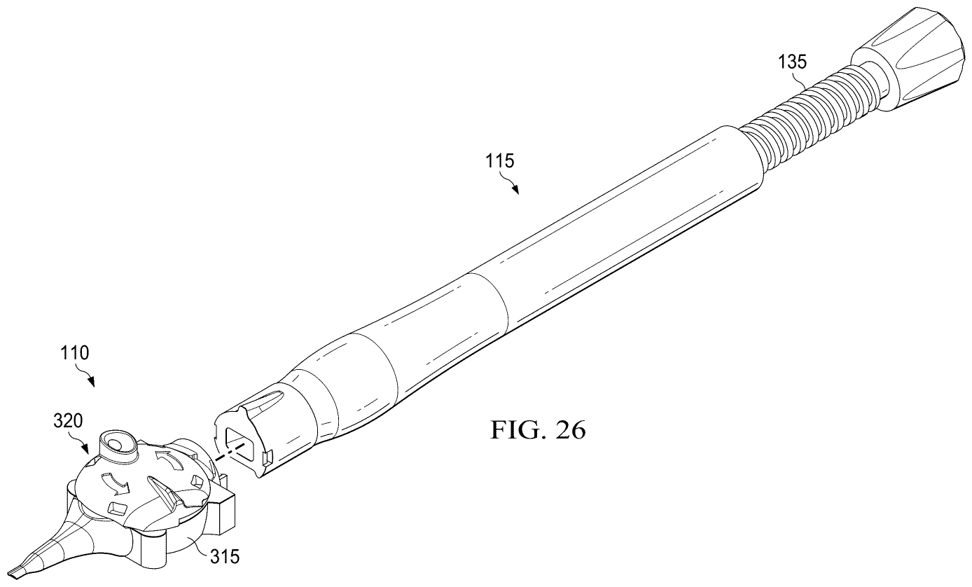

In some embodiments, the transition between the first state illustrated in FIGS. 13, 14, and 15 to the second state illustrated in FIGS. 16 and 17 may illustrate one example of part of a manufacturing and/or assembly process. For example, the various components of the apparatus 100 with the implant management system 110 may be assembled into a single device (or into a cartridge as described with reference to FIGS. 25 and 26), including placement of the implant 1400 within the implant management system 110, in the first state of assembly. For example, the alignment of components illustrated in FIGS. 13, 14, and 15 permits the dial 320 to be placed on the implant support 325 with the alignment of the tabs 1220 with the tab windows 1225 after the implant 1400 is placed within the implant management system 110. A human or robotic device may then rotate the dial 320 to arrive in the assembled and locked state which may provide sufficient rotational resistance to substantially reduce or prevent unintentional rotation of the dial 320 during further packing, shipping, and/or distribution.

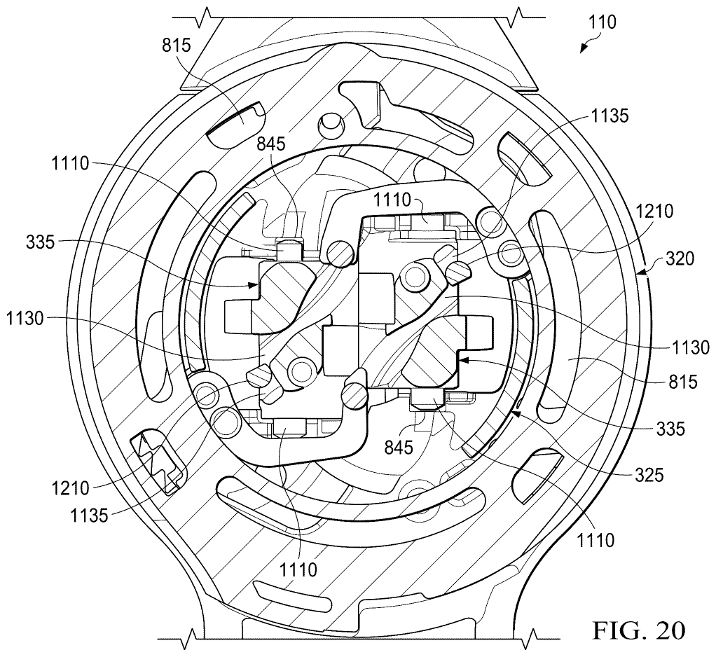

FIG. 18 illustrates the implant management system 110 of FIG. 17 in a third state, in which the dial 320 has been rotated further in the first direction relative to the implant support 325. During the rotation from the second state to the third state, the tabs 1220 (not visible in FIG. 18) continue to rotate behind the tab rail 815, which can provide stability to the dial 320. Although not visible in FIG. 18, rotation of the dial 320 to the third state can also cause the ratchet teeth 1215 to continue to slide past the pawl 825 (see FIG. 16), preventing rotation of the dial 320 in the second direction. In some embodiments, rotation of the dial 320 out of the second state can also move the spring arms 855 out of the recesses 1250. Moving the spring arms 855 out of the recesses 1250 can allow the spring arms 855 to slide along the inside of the ring 1245, reducing the rotational resistance provided by the recesses 1250 in the first state. In the example of FIG. 18, rotation of the dial 320 from the second state to the third state can also cause the dial pins 1010 to travel the length of the folding segment 1235, from the holding segment 1230 to the follower segment 1240. As the dial pins 1010 travel the folding segment 1235, the haptic tab 1015 (not visible in FIG. 18) of each of the folding arms 330 can fold the leading haptic 1410 and the trailing haptic 1415 over the optic body 1405. In some configurations, the dial pins 1010 can be moved substantially simultaneously. For example, the symmetrical configuration of the arm tracks 1205 of FIG. 18 can cause the dial pins 1010 to travel the folding segment 1235 at substantially the same time.

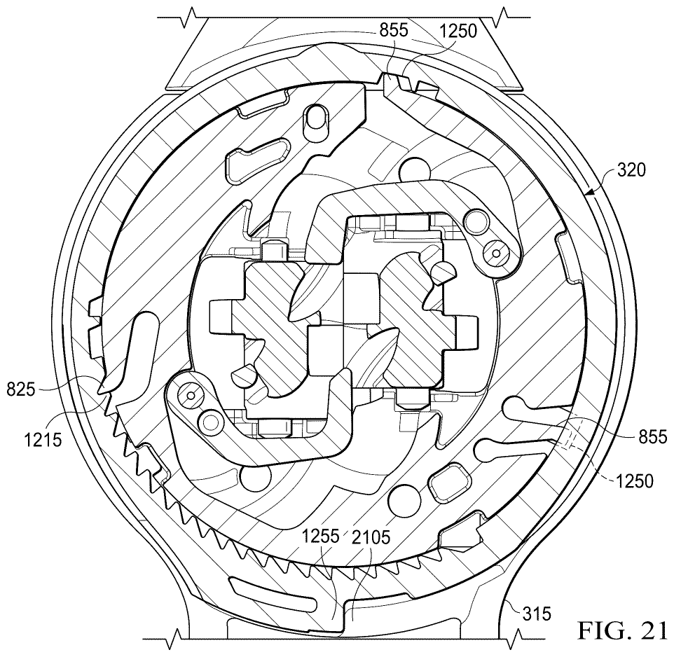

FIG. 19 illustrates the implant management system 110 of FIG. 18, with an additional portion of the dial 320 removed to show additional features that may be associated with some embodiments. For example, in this third state of the dial 320, the roller pins 1210 may engage the folding segment 1130 of the roller track 1120 in each of the edge rollers 335.

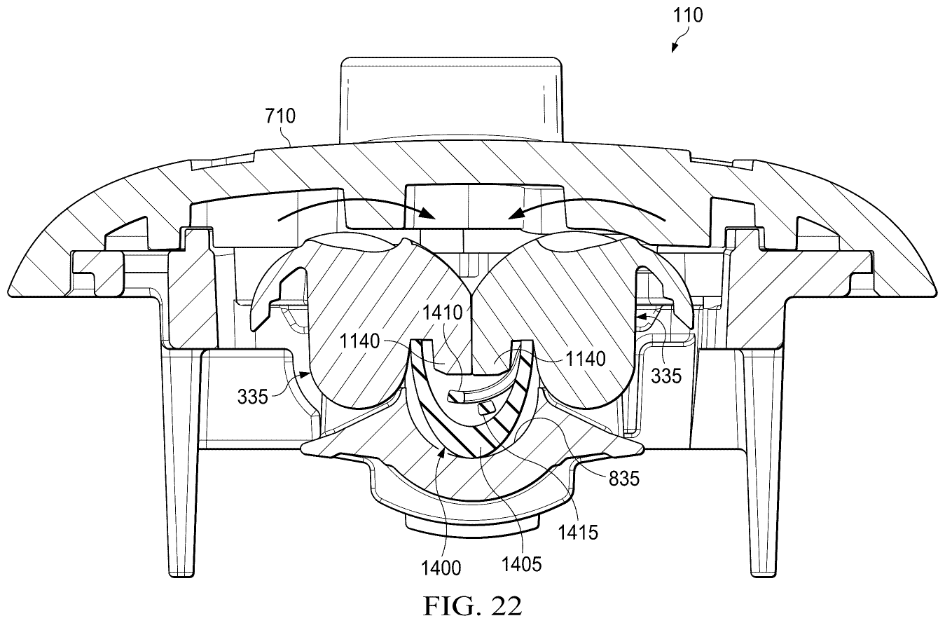

FIG. 20 illustrates the implant management system 110 of FIG. 19 in a fourth state, in which the dial 320 has been rotated further in the first direction relative to the implant support 325. During the rotation from the third state to the fourth state of FIG. 20, the tabs 1220 (not visible in FIG. 20) continue to rotate behind the tab rail 815, and the roller pins 1210 may travel the folding segment 1130 of each of the edge rollers 335. As the roller pins 1210 travel the folding segment 1130, the roller pins 1210 can cause the edge rollers 335 to rotate about the pins 1110 within the pin slots 845, toward the delivery channel 835 (see FIG. 8) behind the optic body 1405 (not visible in FIG. 20). In some embodiments, the action of the roller pins 1210 may initially overcome the resistance of the clip 1115 (see FIG. 11), causing the clip 1115 to disengage the roller tabs 850 (see FIG. 8), allowing further travel of the roller pins 1210 to rotate the edge rollers 335. In some configurations, the edge rollers 335 can be rotated substantially simultaneously. For example, the symmetrical configuration of the roller pins 1210 of FIG. 20 can cause the roller pins 1210 to travel the folding segment 1130 at substantially the same time. Additionally, or alternatively, the orientation and profile of the folding segment 1130 can provide a smooth, linear rotation of the edge rollers 335. In some embodiments, the dial pins 1010 may travel the follower segment 1240 (see, e.g., FIG. 18) as the roller pins 1210 travel the folding segment 1130 to allow the edge rollers 335 to rotate without rotating the folding arms 330. The roller pins 1210 may engage the follower segment 1135 in this fourth state, as illustrated in the example of FIG. 20.

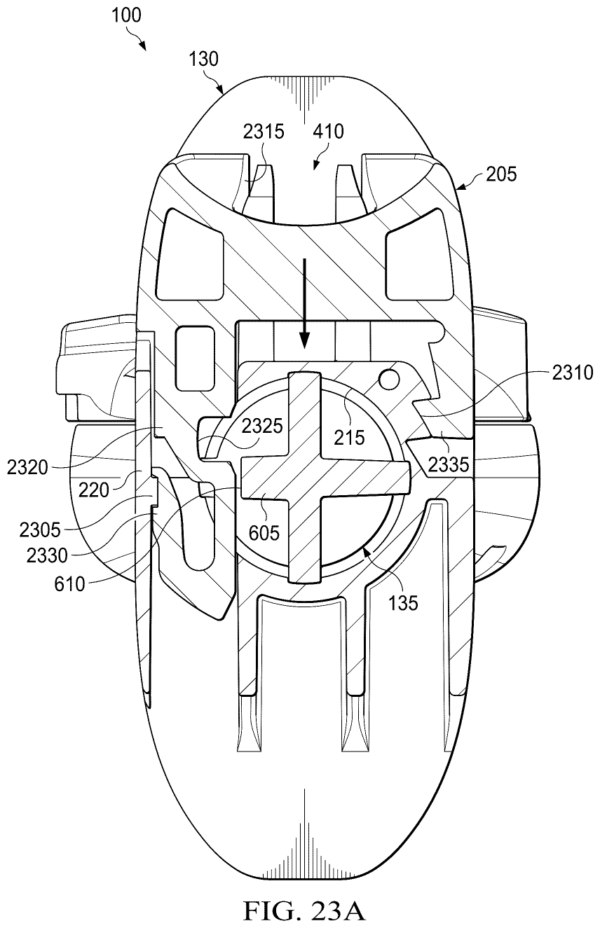

FIG. 21 illustrates the implant management system 110 in the fourth state of FIG. 20, in which an additional portion of the dial 320 has been removed to illustrate further details that may be associated with some embodiments. For example, the stop tab 1255 of FIG. 21 is in contact with a stop 2105, which may be coupled to the base 315 in some embodiments. Contact between the stop tab 1255 and the stop 2105 can prevent further rotation of the dial 320 in the first direction. As also illustrated in the example of FIG. 21, the ratchet teeth 1215 have been rotated so that the pawl 825 is engaged with the last tooth. Additionally, as shown in the example of FIG. 21, one or more of the spring arms 855 may spring into one of the recesses 1250.

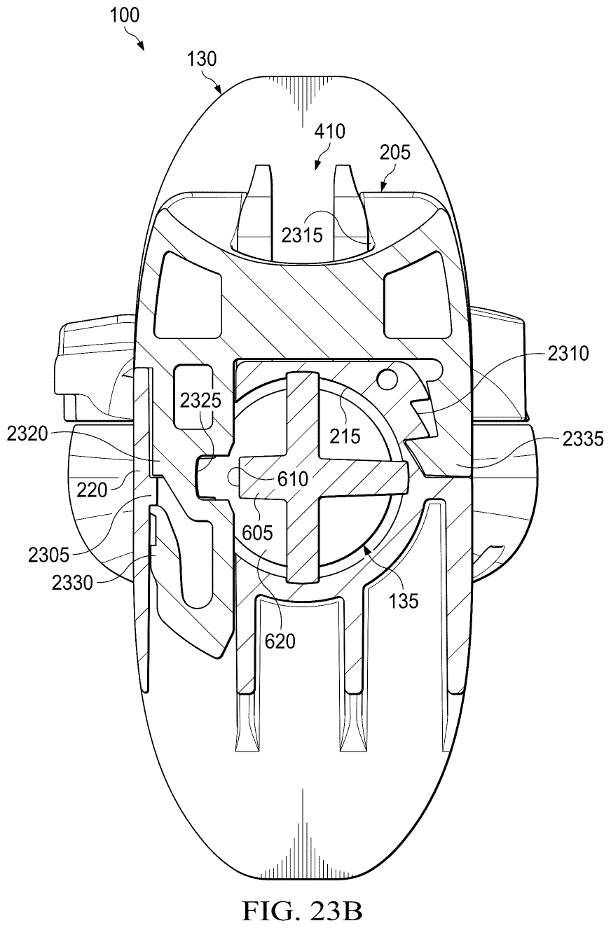

FIG. 22 is a section view of the implant management system 110 in the fourth state, illustrating additional details that may be associated with some embodiments. For example, as in FIG. 20 and FIG. 21, the edge rollers 335 have been rotated, which can cause the folding edge 1140 of each of the edge rollers 335 to fold the optic body 1405 (with the leading haptic 1410 and the trailing haptic 1415) from the edges, inward on itself and into the delivery channel 835. In the example of FIG. 22, the implant 1400 is disposed in the delivery channel 835. The cap 710 of FIG. 22 illustrates an example having a dome shape, which can provide sufficient clearance for rotation of the edge rollers 335.

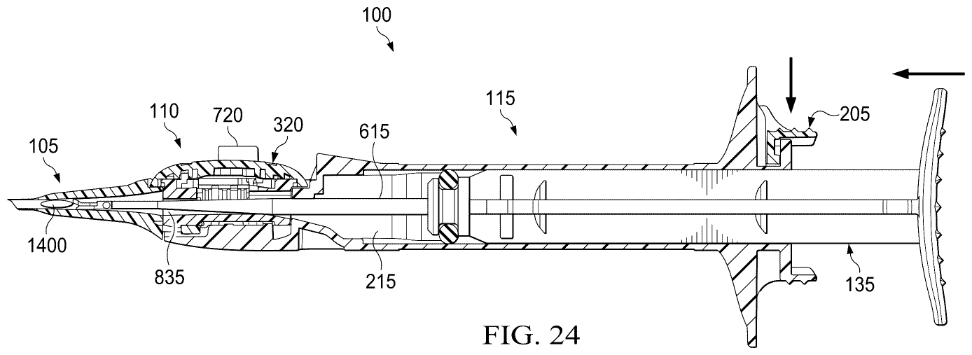

FIG. 23A and FIG. 23B are section views of the apparatus 100 of FIG. 3, taken along section line 23-23. FIG. 23A illustrates additional details that may be associated with some embodiments of the apparatus 100 in which the plunger key 205 is in the locked state of FIG. 3, and FIG. 23B illustrates additional details that may be associated with some embodiments of the apparatus 100 in which the plunger key 205 is in an unlocked state.

In the example of FIG. 23A and FIG. 23B, the key track 410 comprises a pair of parallel track rails, which are generally oriented orthogonal to the bore 215. FIG. 23A and FIG. 23B also illustrate an embodiment of the housing 130 having a first catch 2305 and a second catch 2310, and an embodiment of the plunger key 205 comprising a key guide 2315 and an arm 2320. In the example of FIG. 23A, the key guide 2315 is configured to slidingly engage the key track 410.

The key track 410 can allow the plunger key 205 to move in a first direction toward the bore 215 while substantially preventing lateral movement of the plunger key 205 relative to the key track 410. The arm 2320 of FIG. 23A is disposed through the keyway 610 of the plunger 135, and movement of the key guide 2315 relative to the key track 410 can also move the arm 2320 through the keyway 610 from a first position, illustrated in the example of FIG. 23A, to a second position, illustrated in the example of FIG. 23B.

In the first position, the arm 2320 is configured to block the rail 605. More particularly, the arm 2320 can pass through the keyway 610 so that a rail slot 2325 is offset from the rail 605, thereby preventing the rail 605 (and, thus, the plunger 135) from moving through the rail slot 2325 relative to the bore 215. In the context of the example of FIG. 23A and FIG. 23B, the first position is a locked position.

In the second position of FIG. 23B, the rail slot 2325 is aligned with the rail 605 so that the rail 605 can move through the rail slot 2325, thereby allowing the rail 605 and the plunger 135 to move relative to the bore 215. In the context of the example of FIG. 23A and FIG. 23B, the second position is an unlocked position. In the unlocked position, the arm 2320 may also be configured to block the stop 620 in some embodiments, as illustrated in the example of FIG. 23B. In the unlocked position, the arm 2320 can allow bidirectional movement of the plunger 135 within the bore 215, while the stop 620 can limit such movement in one direction, thereby preventing the plunger 135 from being completely removed from the bore 215.

In the example of FIG. 23A and FIG. 23B, the plunger key 205 also comprises a first snap fitting 2330 and a second snap fitting 2335. The first snap fitting 2330 may be movable with the arm 2320 from the first position to the second position. In some embodiments, the first snap fitting 2330 may be configured to be coupled to the housing 130 in the second position to prevent return of the arm 2320 from the second position toward the first position, thereby maintaining alignment of the rail slot 2325 with the rail 605 and substantially reducing or eliminating the risk of inadvertent locking of the plunger 135 after it is unlocked. In more particular embodiments, the first snap fitting 2330 may be configured to engage the first catch 2305 to prevent return of the arm 2320. Additionally, or alternatively, the first snap fitting 2330 may be configured to be coupled to the housing 130 in the first position to prevent movement of the arm 2320 in a second direction, such as away from the bore 215 in the example of FIG. 23A, which can substantially reduce or eliminate the risk of inadvertent removal of the arm 2320 from the keyway 610. More generally, the first snap fitting 2330 may be configured to be coupled to the housing 130 in the first position, the second position, or both, which can substantially reduce or eliminate the risk of inadvertent removal of the plunger key 205 from the housing 130.

Additionally, or alternatively, in some embodiments, the second snap fitting 2335 may be configured to prevent the arm 2320 from returning to the first position from the second position. For example, the second snap fitting 2335 of FIG. 23B is configured to engage the second catch 2310 to prevent movement of the arm 2320 away from the second position. Some embodiments of the second snap fitting 2335 may also be configured to be coupled to the housing 130 in the first position to prevent movement of the arm 2320 in the second direction, such as away from the bore 215 in the example of FIG. 23A. For example, the second catch 2310 may comprise more than one undercut or stop, and the second snap fitting 2335 may be configured to engage a first stop in the first position and a second stop in the second position.

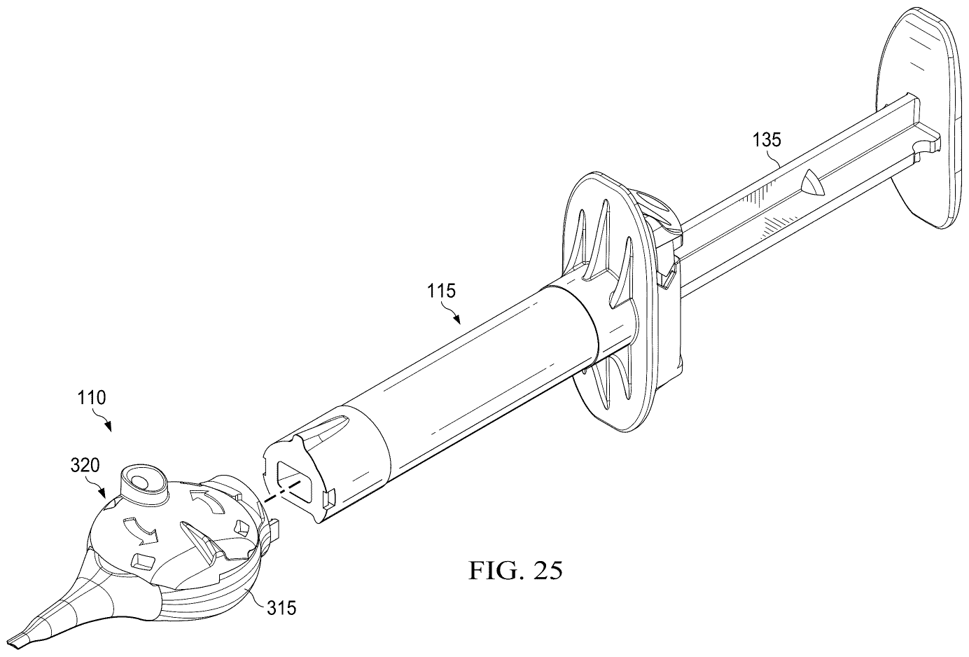

FIG. 24 is a schematic diagram illustrating an example method of using the apparatus 100 to eject the implant 1400. Initially, various components of the system may be assembled if needed or appropriate. In the example of FIG. 24, the nozzle 105, the implant management system 110, and the actuator 115 are fixed together to form a unitary structure. In other embodiments, the apparatus 100 may comprise two or more modules, which can be configured to be coupled and decoupled as appropriate for storage, assembly, use, and disposal.

In use, an operator may receive the apparatus 100 with the implant management system 110 in an assembled and locked position, such as described above with reference to FIG. 16 and FIG. 17. The implant 1400 may be pre-loaded in some embodiments of the implant management system 110. For example, the implant 1400 may be loaded as illustrated in FIG. 14, and then rotated to the second state, as illustrated with reference to FIGS. 16 and 17. From this configuration, the dial 320 may be rotated until the stop tab 1255 contacts with the stop 2105, such as generally described above with reference to FIG. 18 through FIG. 22. Rotating the dial 320 can fold the implant 1400 by controlling the timing and sequence of the movement of the folding arms 330 and the edge rollers 335, such as described above with reference to FIG. 18 through FIG. 22. In this position, the fill port 720 is fluidly coupled to the delivery channel 835, and a suitable fluid may be injected through the fill port 720 into the delivery channel 835.

The plunger key 205 can then be placed in the second, unlocked position, substantially as described with reference to FIG. 23B, which allows the plunger 135 to be advanced through the bore 215 toward the nozzle 105, as shown in the example of FIG. 24.

In some embodiments, the implant interface 615 can be configured to contact or otherwise engage the implant 1400 to advance the implant 1400 from delivery channel 835 of the implant management system 110 through the nozzle 105 as the plunger 135 is advanced. As illustrated in the example of FIG. 24, at least a portion of the implant interface 615 may extend into or through the delivery channel 835 of the implant management system 110 and/or the nozzle 105. As the plunger 135 is fully advanced, the implant interface 615 can eject the implant 1400 from the nozzle 105.

FIG. 25 and FIG. 26 are each an isometric view of another example of the apparatus 100, illustrating additional features that may be associated with some embodiments. For example, the nozzle 105, the implant management system 110, and the base 315 may be combined into a cartridge that can be configured to be coupled and decoupled to various embodiments of the actuator 115. In some embodiments, for example, the actuator 115 and the base 315 may comprise a snap fitting or other interlocking mechanism that can allow the base 315 to be attached to and removed from the actuator 115. Some embodiments of the cartridge may be disposable, and the actuator 115 may be disposable or reusable. In some examples, a reusable embodiment of the actuator 115 may be advantageous for sterilization and other maintenance, as well as increasing reusability and reducing environmental impacts. Additionally, or alternatively, the actuator 115 may have various modes of control, operation, and motive force. For example, the plunger 135 of FIG. 25 may be advanced by pushing, and the plunger 135 of FIG. 26 may be advanced by twisting. In some examples, the actuator 115 may be controlled automatically and/or may be powered electrically, electromechanically, or pneumatically.

In some embodiments, the cartridge may be stored, shipped, and/or manufactured separately from the actuator 115. For example, the cartridge may be sanitized and stored in a blister pack during manufacturing while the actuator 115 may be used for multiple procedures and autoclaved between procedures. As another example, when the actuator 115 is disposable, the cartridge may be stored in a same package but a different portion of the package from the actuator 115. The different portions of the package can include the same or different levels or types of sterilization or packaging (e.g., the actuator 115 may be in a larger package with one cover which, after removal, exposes a second portion of the package with a cover that can be removed to access the cartridge). Additionally, or alternatively, the actuator 115 and the cartridge may be in the same package.

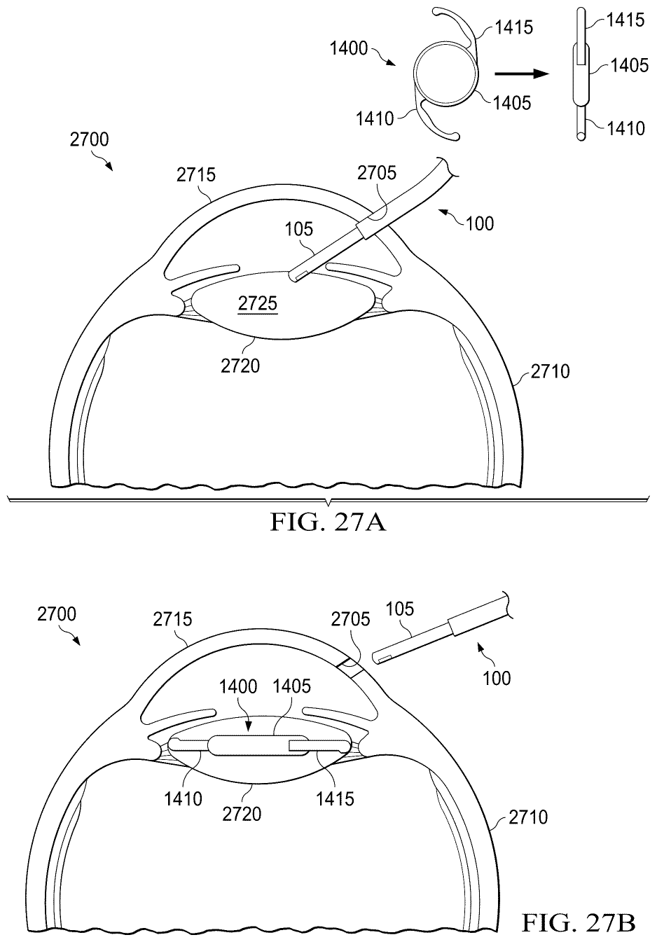

FIG. 27A and FIG. 27B are schematic diagrams illustrating an example use of the apparatus 100 to deliver the implant 1400 to an eye 2700. As illustrated, an incision 2705 may be made in the eye 2700 by a surgeon, for example. In some instances, the incision 2705 may be made through the sclera 2710 of the eye 2700. In other instances, an incision may be formed in the cornea 2715 of the eye 2700. The incision 2705 may be sized to permit insertion of a portion of the nozzle 105 to deliver the implant 1400 into the capsular bag 2720. For example, in some instances, the size of the incision 2705 may have a length less than about 3000 microns (3 millimeters). In other instances, the incision 2705 may have a length of from about 1000 microns to about 1500 microns, from about 1500 microns to about 2000 microns, from about 2000 microns to about 2500 microns, or from about 2500 microns to about 3000 microns.

After the incision 2705 is made, the nozzle 105 can be inserted through the incision 2705 into an interior portion 2725 of the eye 2700. The apparatus 100 can then eject the implant 1400 through the nozzle 105 into the capsular bag 2720 of the eye 2700, substantially as described above.

The implant 1400 may be delivered in a folded configuration and can revert to a resting state with the leading haptic 1410 and the trailing haptic 1415 being at least partially curved around the optic body 1405 within the capsular bag 2720, as shown in FIG. 27B. The capsular bag 2720 can retain the implant 1400 within the capsular bag 2720 in a relationship relative to the eye 2700 so that the optic body 1405 refracts light directed to the retina (not shown). The leading haptic 1410 and the trailing haptic 1415 can engage the capsular bag 2720 to secure the implant 1400 therein. After delivering the implant 1400 into the capsular bag 2720, the nozzle 105 may be removed from the eye 2700 through the incision 2705.

The systems, apparatuses, and methods described herein may provide significant advantages. Some embodiments may be particularly advantageous for delivering intraocular lenses, providing increased reliability and consistency for assembly and implant delivery. For example, some configurations of the implant management system 110 may allow assembly with a single click of the dial, and may allow automated assembly, thereby greatly reducing time and labor required for manufacturing. Additionally, or alternatively, such configurations provide a stable system for storing an implant before delivery.

Some embodiments of the implant management system 110 may additionally, or alternatively, provide reliable and consistent manipulation of an implant prior to delivery, which can greatly improve the success rate of treatment. For example, some configurations of the implant management system 110 can greatly simplify the process of folding an implant before delivery, allowing an operator to reliably and consistently fold the implant with the turn of a dial.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations, the nozzle 105, the implant management system 110, and the actuator 115 may each be separated from one another or combined in various ways for manufacture or sale.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. An apparatus for managing an implant to be delivered to an eye, the apparatus comprising:
an implant support comprising an implant bay, a tab rail, a tab slot, and a pawl;
a folding arm configured to be coupled to the implant support and comprising a dial pin;
an edge roller configured to be coupled to the implant support and comprising a roller track; and
a dial comprising an arm track, a roller pin, a plurality of ratchet teeth, and a tab;
wherein the tab is configured to be aligned with the tab slot so that the arm track engages the dial pin, the roller pin engages the roller track, and the dial is configured to be rotated to move the tab behind the tab rail and to engage the ratchet teeth with the pawl.

2. The apparatus of claim 1, wherein:
the tab rail is disposed around the implant bay; and
the tab slot and the pawl are substantially co-planar with the tab rail.

3. The apparatus of claim 1, wherein:
the dial further comprises a first recess and a second recess;
the implant support further comprises a spring arm configured to be aligned with the first recess; and
rotating the dial moves the spring arm from the first recess to the second recess simultaneously with moving the tab behind the tab rail.

4. The apparatus of claim 1, wherein:
the dial further comprises a first recess and a second recess;
the implant support further comprises a spring arm configured to be aligned with the first recess; and
rotating the dial moves the spring arm from the first recess to the second recess simultaneously with the pawl engaging a first tooth of the ratchet teeth.

5. The apparatus of any preceding claim, wherein:
the dial further comprises a cap having a tab window; and
the tab is aligned with the tab window.

6. The apparatus of any preceding claim, wherein:
the implant is an intraocular lens having an optic body and a haptic;
the folding arm is engaged with the haptic; and
the edge roller is engaged with the optic body.

7. An apparatus for eye surgery, the apparatus comprising:
an implant support comprising an implant bay, a tab rail, a plurality of tab slots, and a pawl;
an implant disposed in the implant bay, the implant comprising an optic body, a first haptic coupled to the optic body, and a second haptic coupled to the optic body;
a first folding arm coupled to the implant support, the first folding arm comprising a first dial pin and a first haptic tab, the first haptic tab configured to engage the first haptic;
a second folding arm coupled to the implant support, the second folding arm comprising a second dial pin and a second haptic tab, the second haptic tab configured to engage the second haptic;
a first edge roller configured to be coupled to the implant support, the first edge roller comprising a first roller track and a first folding edge, the first folding edge configured to engage a first portion of the optic body;
a second edge roller configured to be coupled to the implant support, the second edge roller comprising a second roller track and a second folding edge, the second folding edge configured to engage a second portion of the optic body; and
a dial comprising a ring, a first arm track, a second arm track, a first roller pin, a second roller pin, a plurality of ratchet teeth, and a plurality of tabs;
wherein the ring circumscribes the tab rail, each of the tabs is configured to be aligned with one of the tab slots so that the first arm track engages the first dial pin, the second arm track engages the second dial pin, the first roller pin engages the first roller track, the second roller pin engages the second roller track, and the dial is configured to be rotated to move the tabs behind the tab rail and to engage a first tooth of the ratchet teeth with the pawl.

8. The apparatus of claim 7, wherein:
the tab rail is disposed around the implant bay; and
the tab slots and the pawl are substantially co-planar with the tab rail.

9. The apparatus of claim 7, wherein:
the dial further comprises a plurality of recesses;
the implant support further comprises a first spring arm and a second spring arm; and
each of the pawl, the first spring arm, and the second spring arm are configured to be aligned with one of the recesses.

10. The apparatus of claim 9, wherein:
the dial is further configured to be rotated to move the first spring arm and the second spring arm to an adjacent recess substantially simultaneously with the first tooth engaging the pawl.

11. The apparatus of claim 9, wherein:
the tab rail is disposed around the implant bay;
the tab slots, the pawl, the first spring arm, and the second spring arm are substantially co-planar with the tab rail; and
the pawl, the first spring arm, and the second spring arm are equidistantly spaced around the implant support.

12. The apparatus of claim 9, wherein:
the dial further comprises a cap having a plurality of tab windows; and
each of the tabs is aligned with one of the tab windows.

13. The apparatus of any of claims 7-12, further comprising:
a base coupled to the implant support;
a nozzle coupled to the base; and
an actuator configured to advance the implant from the implant support to the nozzle.

14. The apparatus of any of claims 7-12, further comprising:
a base coupled to the implant support; and
a nozzle coupled to the base.

15. The apparatus of any of claims 7-12, further comprising:
a base coupled to the implant support;
a nozzle coupled to the base; and
an actuator configured to be coupled to the base and operable to advance the implant from the implant support to the nozzle if coupled to the base.

16. A method for assembling an apparatus for eye surgery, the method comprising:
receiving an implant support comprising an implant bay, a tab rail, a tab slot, and a pawl;
receiving a folding arm comprising a dial pin;
receiving an edge roller comprising a roller track;
receiving a dial comprising an arm track, a roller pin, a plurality of ratchet teeth, and a tab;
coupling the folding arm and the edge roller to the implant support;
aligning the tab with the tab slot so that the arm track engages the dial pin and the roller pin engages the roller track; and
rotating the dial to simultaneously move the tab behind the tab rail and engage the ratchet teeth with the pawl.

17. The method of claim 16, wherein:
the dial further comprises a first recess and a second recess;
the implant support further comprises a spring arm configured to be aligned with the first recess; and
rotating the dial simultaneously moves the spring arm from the first recess to the second recess.

18. The method of claim 17, wherein rotating the dial engages a first tooth of the ratchet teeth.

19. The method of claim 18, wherein:
the dial further comprises a cap having a tab window; and
the tab is aligned with the tab window.

20. The method of any of claims 16-19, further comprising:
coupling the implant support to a base; and
coupling the base to an actuator configured to advance an implant from the implant bay.

21. The systems, apparatuses, and methods substantially as described herein.
